(19) United States
(12) Patent Application Publication
(10) Pub. No.: US 2020/0390161 A1
(43) Pub. Date: Dec. 17, 2020

(54) Undergarment

(71) Applicant: VBrite, LLC, Rogers, AR (US)

(72) Inventor: Shyhshin Liou, Rogers, AR (US)

(21) Appl. No.: 16/437,100

(22) Filed: Jun. 11, 2019

(51) Int. Cl. A41B 9/00; A61F 13/505; A61F 13/84; A61F 13/496

(52) U.S. Cl. CPC A41B 9/001; A61F 13/505; A61F 13/8405; A61F 13/496; A41B 9/02; A41B 9/004; A61F 2013/8435; D10B 2401/13; A41B 2400/34

(57) ABSTRACT

An undergarment has a fabric body having a base layer and a lining layer. The lining layer is adjacent to an inner side of the base layer and has an odor controlling and antimicrobial layer being away from the base layer and a leak-proof layer being adjacent to the base layer. The odor controlling and antimicrobial layer has a bamboo charcoal fiber layer and a coffee ground fabric layer. The undergarment has advantages of moisture absorption, leak-proofness, odor control, and antimicrobial function.

### BACKGROUND OF THE INVENTION

### 1. Field of the Invention

The present invention relates to an undergarment, and more particularly to an undergarment that is odor controlling, antimicrobial and leak-proof.

### 2. Description of Related Art

Undergarments, such as underpants, drawers, shorts, etc., are worn beneath outer garments, and are usually in direct contact with the skin. The undergarments are moisture absorbing to prevent the outer garments from getting wet.

However, the bodily fluids, such as those associated with leakage of urine, vaginal discharge, and bulbourethral gland fluid, may not be absorbed by the conventional undergarment completely, and will leak out from the conventional undergarment to wet, stain and soil the outer garments. Moreover, the conventional undergarment is smelly and germ breeding after absorbing the bodily fluids.

To overcome the shortcomings, the present invention tends to provide an undergarment to mitigate or obviate the aforementioned problems via plant infused solution.

### SUMMARY OF THE INVENTION

The main objective of the invention is to provide an undergarment that is moisture absorbing, leak-proof, odor controlling, and antimicrobial.

The undergarment has a waist opening, a pair of leg openings, and a fabric body. The fabric body has a base layer and a lining layer. The lining layer is adjacent to an inner side of the base layer and has an odor controlling and antimicrobial layer being away from the base layer and a leak-proof layer being adjacent to the base layer. The odor controlling and antimicrobial layer has a bamboo charcoal fiber layer made of a fabric containing bamboo charcoal fibers and a coffee ground fabric layer made of a fabric containing coffee grounds.

Other objects, advantages and novel features of the invention will become more apparent from the following detailed description when taken in conjunction with the accompanying drawings.

### BRIEF DESCRIPTION OF THE DRAWINGS

FIG. 1 is a front view of an undergarment in accordance with the present invention;

FIG. 2 is an exploded perspective view of a first embodiment of layers of a fabric body of the undergarment in FIG. 1;

FIG. 3 is an enlarged cross sectional side view of the fabric body of the undergarment in FIG. 2;

FIG. 4 is an enlarged front view of the bamboo charcoal fiber layer in FIG. 2;

FIG. 5 is an enlarged cross sectional side view of the bamboo charcoal fiber layer in FIG. 4;

FIG. 6 is an enlarged front view of the coffee ground fabric layer in FIG. 3; and

FIG. 7 is an exploded perspective view of a second embodiment of layers of a fabric body of an undergarment in accordance with the present invention.

### DETAILED DESCRIPTION OF PREFERRED EMBODIMENT

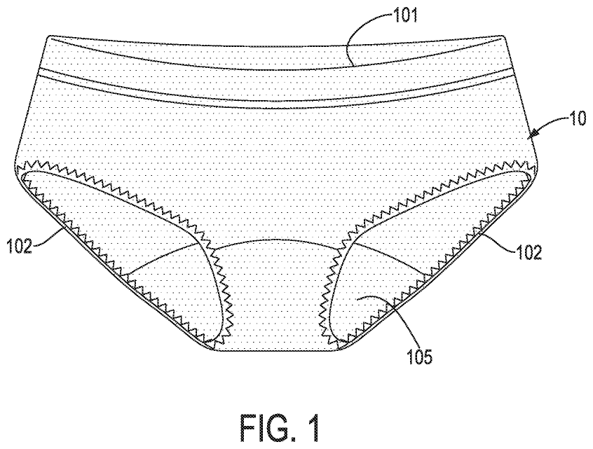

With reference to FIGS. 1 to 3, an undergarment in accordance with the present invention comprises a fabric body 10, a waist opening 101, and a pair of leg openings 102. The fabric body 10 is defined between the waist opening 101 and the pair of leg openings 102 and has a crotch region 105 between the pair of the leg openings 102. The fabric body 10 includes a base layer 13 and a lining layer 20. The base layer 13 faces to the outer garments worn by the wearer, and is defined in an entirety of the fabric body 10. The lining layer 20 is defined in the inner side of the fabric body 10, which faces to the skin of the wearer, and is adjacent to an inner side of the base layer 13. The lining layer 20 may be defined in an entirety or a partial region of the fabric body 10. For example, the lining layer 20 may be only defined in the crotch region 105 of the fabric body 10, may extend over the crotch region 105 of the fabric body 10, or extend to the entirety of the fabric body 10.

The base layer 13 may contain organic cotton, nylon, elastane, breathable co-polyester fabric, or any combination thereof.

The lining layer 20 includes an odor controlling and antimicrobial layer 21 and a leak-proof layer 23. The odor controlling and antimicrobial layer 21 is away from the base layer 13, and the leak-proof layer 23 is adjacent to the base layer 13. The odor controlling and antimicrobial layer 21 includes a bamboo charcoal fiber layer 211 and a coffee ground fabric layer 212.

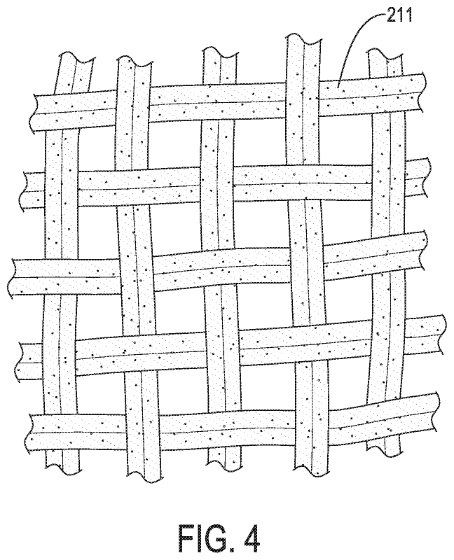

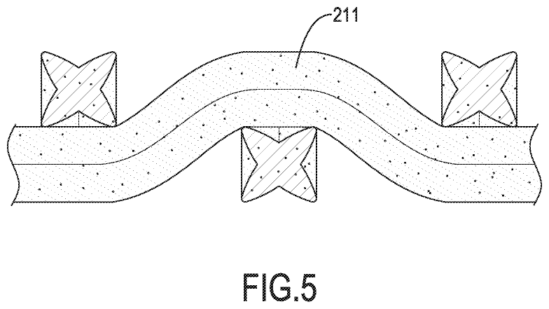

With reference to FIGS. 4 and 5, the bamboo charcoal fiber layer 211 is a fabric containing bamboo charcoal fibers. The manufacturing steps of the bamboo charcoal fiber layer 211 may be as follows.
1. Mix carbonizing bamboo grains and polyester grains to manufacture master batches, and the master batches may contain 2.5% of carbonizing bamboo grains and 97.5% of polyester grains.
2. Draw yarns from the master batches.
3. Manufacture the fabric containing bamboo charcoal fibers from the yarns.

With such manufacturing steps, the carbonized bamboo particles are embedded in the fabric rather than simply coated onto the surface, so the fabric can be washed numerous times with no adverse effect on quality. The bamboo charcoal fiber layer 211 has the following advantages: bacterial metabolism inhibition, antimicrobial function, deodorizing ability, moisture absorption, ventilation, and far infrared radiation properties. The far infrared radiation properties can increase cellular energy, accelerate blood circulation speed, and promote body metabolism. In addition, the crisscross shaped fiber can minimize discomfort through reducing skin contact with bodily fluids.

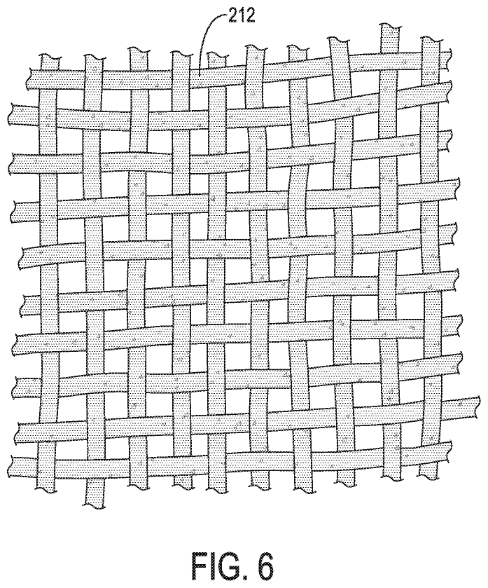

With reference to FIG. 6, the coffee ground fabric layer 212 is a fabric containing coffee grounds. The coffee grounds may be made from baked coffee residues or microencapsulated baked coffee residues. The manufacturing steps of the coffee ground fabric layer 212 may be as follows.
1. Mix carbonizing coffee ground particles to form a mixture.
2. Blend the mixture with polymer chips at a weight ratio of 1:9 to produce master batches.
3. Draw yarns from the master batches.
4. Manufacture the fabric containing coffee grounds from the yarns.

With such manufacturing steps, the carbonized coffee ground particles are embedded in the fabric rather than simply coated onto the surface, so the fabric can be washed numerous times with no adverse effect on quality. The coffee ground fabric layer 212 has the following advantages: fast drying, odor control, and ice cool touch.

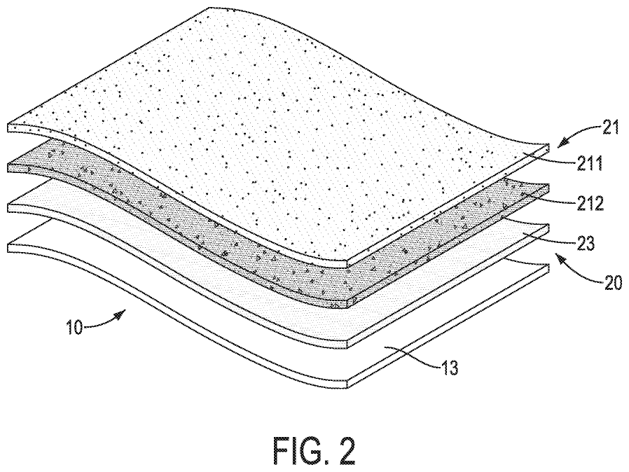

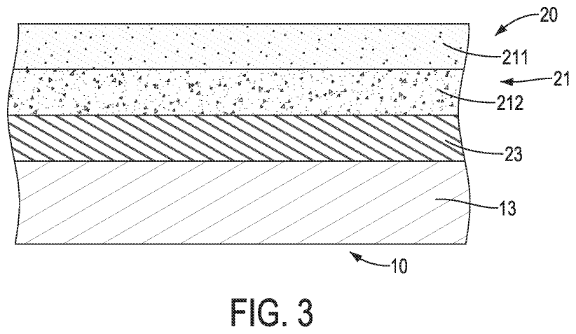

With reference to FIGS. 2 and 3, the leak-proof layer 23 is a leak-proof and breathable fabric. The leak-proof layer 23 may be an elastic cushion having more than 1000 ventilation slits per square inch. Air can permeate through the leak-proof layer 23 and moisture is prevented from leaking out the leak-proof layer 23.

With such arrangements, the bodily fluids can be absorbed by the odor controlling and antimicrobial layer 21. The leak-proof layer 23 can prevent bodily fluids from leaking from the lining layer 20 to outer garments. The outer garments can be prevented from getting wetted, stained and soiled by the bodily fluids. The odor controlling and antimicrobial layer 21 contains a high volume of bamboo charcoal particles and the carbonized coffee ground particles to increase absorption efficiency. The bamboo charcoal particles have the porous structure, can absorb moisture, prevent bacteria growth, reduce odor, and disrupt the germs spread. The carbonized coffee ground particles can sop up hydrogen sulfide gas to absorb odors of the bodily fluids. The carbonized coffee ground particles can increase moisture transferring ability of the coffee ground fabric layer 212 to shorten the duration of the drying process. The coffee ground fabric layer 212 has a cooling feel.

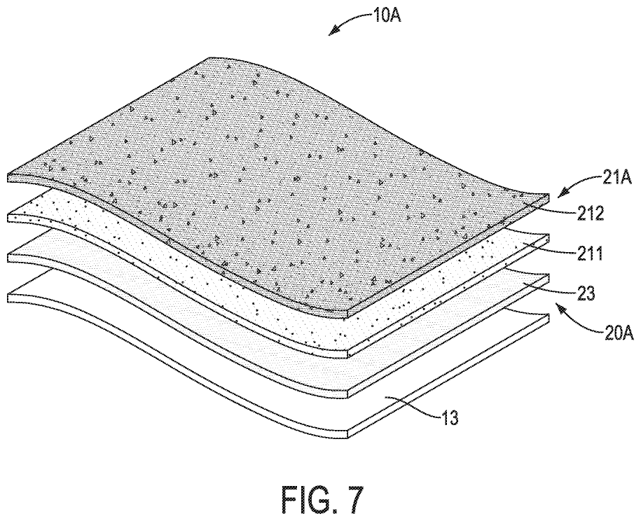

With reference FIG. 2, in the first embodiment of the fabric body 10, the bamboo charcoal fiber layer 211 is away from the leak-proof layer 23, and the coffee ground layer 212 is adjacent to the leak-proof layer 23. The bamboo charcoal fiber layer 211 is placed near the skin of the wearer to increase far infrared radiation properties and the insulating ability can reduce heat lost, making the undergarment a perfect choice for cold days. With reference FIG. 7, in the second embodiment of the fabric body 10A, the coffee ground layer 212 is away from the leak-proof layer 23, and the bamboo charcoal fiber layer 211 is adjacent to the leak-proof layer 23. The coffee ground layer 212 is placed near the skin of the wearer to enhance the cool feel and to relieve discomfort during hot summer days.

While both bamboo charcoal fiber layer 211 and coffee grounds layer 212 have odor absorption ability, each of them performs at a different rate and one works better than the other on certain types of odors. To maximize results, utilizing both bamboo charcoal fiber layer 211 and coffee grounds layer 212 delivers a superior solution. With the combination of the bamboo charcoal fiber layer 211 and the coffee grounds layer 212, the fabric body 10 can reduce about 75.6% ammonia (NH₃) gas after the determination of deodorant property by detector tube method (ISO 17299-2:2014). The odor controlling ability of the fabric body 10 is better than the odor controlling and antimicrobial layer 21 that only has the bamboo charcoal fiber layer 211 or the coffee ground layer 212. The undergarment in accordance with the present invention has advantages of moisture absorption, leak-proofness, odor control, and antimicrobial function.

What is claimed is:

1. An undergarment comprising:
a waist opening;
a pair of leg openings; and
a fabric body defined between the waist opening and the pair of leg openings and having
a base layer; and
a lining layer being adjacent to an inner side of the base layer and having
an odor controlling and antimicrobial layer being away from the base layer and having
a bamboo charcoal fiber layer made of a fabric containing bamboo charcoal fibers; and
a coffee ground fabric layer made of a fabric containing coffee grounds; and
a leak-proof layer being adjacent to the base layer.

2. The undergarment as claimed in claim 1, wherein
the bamboo charcoal fiber layer of the odor controlling and antimicrobial layer is away from the leak-proof layer; and
the coffee ground fabric layer of the odor controlling and antimicrobial layer is adjacent to the leak-proof layer.

3. The undergarment as claimed in claim 1, wherein
the bamboo charcoal fiber layer of the odor controlling and antimicrobial layer is adjacent to the leak-proof layer; and
the coffee ground fabric layer of the odor controlling and antimicrobial layer is away from the leak-proof layer.

4. The undergarment as claimed in claim 1, wherein the lining layer is defined in an entirety of the fabric body.

5. The undergarment as claimed in claim 2, wherein the lining layer is defined in an entirety of the fabric body.

6. The undergarment as claimed in claim 3, wherein the lining layer is defined in an entirety of the fabric body.

7. The undergarment as claimed in claim 1, wherein the lining layer is defined in a partial region of the fabric body.

8. The undergarment as claimed in claim 2, wherein the lining layer is defined in a partial region of the fabric body.

9. The undergarment as claimed in claim 3, wherein the lining layer is defined in a partial region of the fabric body.

10. The undergarment as claimed in claim 7, wherein
the fabric body has a crotch region defined between the pair of the leg openings; and
the lining layer is defined in the crotch region of the fabric body.

11. The undergarment as claimed in claim 8, wherein
the fabric body has a crotch region defined between the pair of the leg openings; and
the lining layer is defined in the crotch region of the fabric body.

12. The undergarment as claimed in claim 9, wherein
the fabric body has a crotch region defined between the pair of the leg openings; and
the lining layer is defined in the crotch region of the fabric body.
